(19) United States
(12) Patent Application Publication
(10) Pub. No.: US 2026/0258187 A1
(43) Pub. Date: Sep. 3, 2026

(54) Method for Preparing Isocyanurate-Group-Containing Polyisocyanates

(71) Applicant: Covestro Deutschland AG, Leverkusen (DE)

(72) Inventors: Esther Kaese, Leverkusen (DE); Alejandro Munera Parra, Leverkusen (DE); Johannes Kiecherer, Köln (DE); Leonie Golka, Köln (DE); Frank Behrendt, Langenfeld (DE); Alicia Heilmann, Radevormwald (DE)

(21) Appl. No.: 19/489,444

(22) PCT Filed: Jun. 10, 2024
(86) PCT No.: PCT/EP2024/065924

§ 371 Date: Dec. 2, 2025

(30) Foreign Application Priority Data
Jun. 28, 2023 (EP) 23182104.2

(51) Int. Cl. C08G 18/09; C08G 18/18; C08G 18/28; C08G 18/32

(52) U.S. Cl. CPC C08G 18/092; C08G 18/1875; C08G 18/2825; C08G 18/3206; C08G 2115/02

(57) ABSTRACT

The invention relates to a process for preparing isocyanurate group-containing polyisocyanates P by trimerizing
A) at least one organic di- or polyisocyanate having independently aliphatically, cycloaliphatically and/or araliphatically bonded isocyanate groups,
in the presence of
B) at least one trimerization catalyst selected from the group consisting of quaternary tetraalkylammonium hydroxides, quaternary trialkylarylammonium hydroxides and hydroxyalkyl-substituted quaternary ammonium hydroxides of choline type
and
C) at least one alcohol as solvent.

### CROSS-REFERENCE TO RELATED APPLICATIONS

This application is the United States national phase of International Patent Application No. PCT/EP2024/065924 filed Jun. 10, 2024, and claims priority to European Patent Application No. 23182104.2 filed Jun. 28, 2023, the disclosures of which are hereby incorporated by reference in their entireties.

### BACKGROUND

### Technical Field

The preparation of isocyanurate group-containing polyisocyanates by trimerizing monomeric di- and/or polyisocyanates has long been known.

Use is usually made of trimerization catalysts, for example quaternary tetraalkyl- or trialkylarylammonium hydroxides, such as Triton-B and hydroxyalkyl-substituted quaternary ammonium hydroxides of choline type, for example choline acetate.

### Description of Related Art

The known processes in the prior art (for example EP 2 700 665 A1) are carried out in such a way that an alcoholic solution of the catalyst is first fed to the isocyanate component to be trimerized. The resultant start of the exothermic reaction increases the temperature of the reaction mixture. The exothermicity is controlled during the further course of the process by adding adapted amounts of the catalyst solution.

It has now surprisingly been found that it is possible to improve the reaction behavior if, after starting the reaction by way of the initial addition of an alcoholic catalyst solution, only alcohol, but no further catalyst, is subsequently metered in. This metering behavior results in less catalyst being consumed while the reaction time remains the same, or in it being possible for the reaction time to be shortened for semibatch processes due to a quicker reaction start, and for the throughput for cascade processes to be increased, while the amount of catalyst remains the same. In the first case, the lower consumption of catalyst also means that less stopper is needed to stop the reaction, and less catalyst is present in the product, which improves the product quality (less tendency to discoloration). A reduced reaction time means a more economically viable process. In addition, the reaction regime can be better adapted to the quality of the diisocyanate used (in relation to acidic components), since added amounts of catalyst and alcohol can be controlled independently of each other.

### SUMMARY

The invention provides a process for preparing isocyanurate group-containing polyisocyanates P by trimerizing
A) at least one organic di- or polyisocyanate having independently aliphatically, cycloaliphatically and/or araliphatically bonded isocyanate groups, in the presence of
B) at least one trimerization catalyst selected from the group consisting of quaternary tetraalkylammonium hydroxides, quaternary trialkylarylammonium hydroxides and hydroxyalkyl-substituted quaternary ammonium hydroxides of choline type
and
C) at least one alcohol as solvent,
comprising or consisting of the following steps
   I) initially charging component A into a reactor,
   II) feeding the total amount of catalyst component B, and a first partial amount of component C (CT1), and trimerizing component A until a degree of trimerization T_{g} in the range of ≥0.5% to ≤25%, preferably ≥0.5% to ≤20%, is obtained, where

$${T}_{g} = ( {NCO}_{0} - {NCO}_{t} ) / {NCO}_{0} ,$$

where
NCO₀ corresponds to the amount of free NCO groups originally present in the initially charged component A and
NCOₜ corresponds to the amount of free NCO groups in the reaction solution at time t, in each case determined by NCO titration in accordance with M105-ISO 11909,
III) feeding a second partial amount of component C (C_{T2}), continuing the trimerization of component A.

The above process is referred to hereinafter as embodiment 1.

It is also possible to add the alcoholic catalyst solution initially in a high concentration and then to subsequently meter in the catalyst solution at only a low concentration after the start of the reaction.

The invention therefore also provides a process for preparing isocyanurate group-containing polyisocyanates P′ by trimerizing
A′) at least one organic di- or polyisocyanate having independently aliphatically, cycloaliphatically and/or araliphatically bonded isocyanate groups, in the presence of
B′) at least one trimerization catalyst selected from the group consisting of quaternary tetraalkylammonium hydroxides, quaternary trialkylarylammonium hydroxides and hydroxyalkyl-substituted quaternary ammonium hydroxides of choline type
and
C′) at least one alcohol as solvent,
comprising or consisting of the following steps
   I′) initially charging component A′ into a reactor,
   II′) feeding a first partial amount of catalyst component B′ (B′_{T1}), and a first partial amount of component C′ (C′_{T1}),
      where catalyst component B′_{T1} is used in amounts of ≥0.6% to ≤8% by weight, preferably ≥0.8% to ≤5% by weight, particularly preferably ≥0.8% to ≤2% by weight, based on the first partial amount of component C′ (C′_{T1}) used in step II′,
      and trimerizing component A′ until a degree of trimerization T_{g}′ in the range of ≥0.5% to ≤25%, preferably of ≥0.5% to ≤20%, is obtained, where

$${T}_{g}^{′} = ( {NCO}_{0} - {NCO}_{t} ) / {NCO}_{0} ,$$

where
NCO₀ corresponds to the amount of free NCO groups originally present in the initially charged component A′ and
NCOₜ corresponds to the amount of free NCO groups in the reaction solution at time t,
in each case determined by NCO titration in accordance with M105-ISO 11909,
III′) feeding a second partial amount of catalyst component B′ (B′_{T2}), and a second partial amount of component C′ (C′_{T2}),
   where catalyst component B′_{T2} is used in amounts of >0% to ≤0.5% by weight, preferably >0% to ≤0.1% by weight, based on the second partial amount of component C′ (C′_{T2}) used in step III′,
   continuing the trimerization of component A′.

This process is referred to hereinafter as embodiment 2.

In the context of the present invention, aliphatic compounds are understood to mean those having exclusively open-chain aliphatic groups, which may be branched or unbranched. Cycloaliphatic compounds are those comprising at least one cycloaliphatic ring system. Araliphatic compounds are those having at least one araliphatic group.

To carry out the processes according to the invention, use may be made of all organic di- or polyisocyanates with an (average) molecular weight of 154-600 g/mol having independently aliphatically, cycloaliphatically and/or araliphatically bonded isocyanate groups, in pure form or as any desired mixtures with one another. Examples include: pentamethylene diisocyanate (PDI), hexamethylene diisocyanate (HDI), 2-methylpentane 1,5-diisocyanate (MPDI), 1,3- and 1,4-bis(isocyanatomethyl)cyclohexane (1,3- and 1,4-H₆-XDI), 1,3- and 1,4-bis(isocyanatomethyl)benzene (1,3- and 1,4-XDI), 3 (4)-isocyanatomethyl-1-methylcyclohexyl isocyanate (IMCI); isophorone diisocyanate (IPDI), bis(isocyanatomethyl) norbornane (NBDI), 4-isocyanatomethyloctane 1,8-diisocyanate (triisocyanatononane, TIN), 1,3-bis(isocyanatomethyl)benzene, 1,3-bis(2-isocyanatopropyl-2) benzene and bis(4 (2)-isocyanatocyclohexyl) methane (H₁₂MDI, Desmodur® W, product of Covestro AG). It is irrelevant here which process is used to prepare the above-mentioned (poly) isocyanates, i.e. with or without use of phosgene.

Preferably, the at least one organic di- or polyisocyanate is selected from the group consisting of PDI, HDI, MPDI, 1,3- and 1,4-H₆XDI, 1,3- and 1,4-XDI and NBDI. Particular preference is given to HDI or a mixture of HDI with PDI, MPDI, 1,3- and 1,4-H₆XDI, 1,3- and 1,4-XDI and/or NBDI.

In step 1 of embodiment 1, component A may be degassed under reduced pressure and optionally with supply of heat. It is preferable to degas component A in step I. The same applies for component A′ in step I′ of embodiment 2.

In the processes according to the invention, at least one quaternary trialkylarylammonium hydroxide and/or at least one hydroxyalkyl-substituted quaternary ammonium hydroxide of choline type is preferably used as catalyst component B or B′. Particularly preferably, at least one benzyltrialkylammonium hydroxide and/or at least one hydroxyalkyl-substituted quaternary ammonium hydroxide of choline type is used. Especially preferably, benzyltrimethylammonium hydroxide (Triton-B) and/or 2-hydroxyethyl-trimethylammonium acetate (choline acetate) is used.

As solvent component C or C′, use is preferably made of any desired aliphatic and/or cycloaliphatic alcohols, preferably aliphatic alcohols, with preference being given to low-molecular-weight mono- or diols. Examples include: methanol, ethanol, isopropanol, n-butanol, 2-ethylhexanol, 2-ethylhexane-1,3-diol, 1,2-dihydroxyethane, 1,2-dihydroxypropane, 1,3- and 1,4-dihydroxybutane, 1,6- and 2,5-dihydroxyhexane, or 2,2,4-trimethyl-1,3-dihydroxypentane or any desired mixtures of these alcohols. Particular preference is given to alcohols having at least one primary alcohol group. Very particular preference is given to n-butanol, 2-ethylhexanol, 2-ethylhexane-1,3-diol, 1,2-dihydroxyethane, 1,2-dihydroxypropane, 1,3- and 1,4-dihydroxybutane, 1,6-dihydroxyhexane, or 2,2,4-trimethyl-1,3-dihydroxypentane or any desired mixtures of these alcohols.

In embodiment 1, the at least one alcohol C used in step II may be identical to or different from the at least one alcohol used in step III. Likewise, in embodiment 2, the at least one alcohol C′ used in step II′ may be identical to or different from the at least one alcohol used in step III′.

### Embodiment 1

In step II, the total amount of catalyst component B and a first partial amount of solvent component C (C_{T1}) are fed to the reactor and component A is trimerized until a degree of trimerization T_{g} in the range of ≥0.5% to ≤25%, preferably of ≥0.5% to ≤20%, is obtained.

The feeding of components B and C is preferably performed in such a way that at least part of the total amount of catalyst component B is dissolved in at least part of the first partial amount of solvent component C. The following embodiments are preferred in this case:

The total amount of component B is fed to the reactor dissolved in the entire first partial amount of component C (embodiment a), where the feeding may be performed continuously or discontinuously.

The total amount of component B is fed to the reactor dissolved in a first part of the first partial amount of component C and the remaining part of the first partial amount of component C is fed to the reactor as pure component C (embodiment b), where both may be performed continuously or discontinuously independently of each other. In this context, the expression “pure component C” means that C does not comprise any catalysts of component B.

Catalyst component B is used in amounts of preferably ≥0.3% to ≤8% by weight, particularly preferably ≥0.5% to ≤5% by weight, very particularly preferably ≥0.8% to ≤2% by weight, in each case based on the total amount of solvent component C used in step II, i.e. based on C_{T1}.

Catalyst component B is generally used in amounts of ≥0.001% to ≤2% by weight, preferably of ≥0.001% to ≤1% by weight and particularly preferably of ≥0.001% to ≤0.2% by weight, in each case based on the amount of isocyanate component A used.

Step II may be carried out in such a way that the trimerization starts already during the feeding or only after the feeding of component B. This may be thermally influenced. The first-mentioned option is preferred.

The trimerization in step II is carried out at reaction temperatures of preferably ≥50° C. to ≤120° C., particularly preferably ≥55° C. to ≤90° C. If HDI is used as component A, the trimerization in step II is very particularly preferably carried out at ≥57° C. to ≤65° C.

In step III, a second partial amount of solvent component C (C_{T2}) is fed to the reactor. This may be performed continuously or discontinuously.

The total amount of solvent component C fed corresponds to the sum total of the two partial amounts from steps II and III (C_{T1}+C_{T2}). The total amount of solvent component C fed is preferably ≥0.3% to ≤5% by weight, particularly preferably ≥1% to ≤2% by weight, in each case based on the amount of isocyanate component A used.

The trimerization in step III is performed at reaction temperatures of preferably ≥50° C. to ≤120° C., particularly preferably ≥55° C. to ≤90° C. If HDI is used as component A, the trimerization in step III is very particularly preferably carried out at ≥60° C. to ≤65° C.

The reaction temperatures in step II and step III may be the same or different.

In step III, the trimerization of component A is continued until the desired degree of trimerization is achieved.

### Embodiment 2

In step II′, a first partial amount of catalyst component B′ (B′_{T1}) and a first partial amount of solvent component C′ (C′_{T1}) are fed to the reactor and component A′ is trimerized until a degree of trimerization T_{g}′ in the range of ≥0.5% to ≤25%, preferably of ≥0.5% to ≤20%, is obtained.

The feeding of components B′ and C′ is preferably performed in this case in such a way that at least part of the first partial amount of catalyst component B′ is dissolved in at least part of the first partial amount of solvent component C′. The following embodiments are preferred in this case:

The entire first partial amount of component B′ is fed to the reactor dissolved in the entire first partial amount of component C′ (embodiment a′), where the feeding may be performed continuously or discontinuously.

The entire first partial amount of component B′ is fed to the reactor dissolved in a first part of the first partial amount of component C′ and the remaining part of the first partial amount of component C′ is fed to the reactor as pure component C′ (embodiment b′), where both may be performed continuously or discontinuously independently of each other. In this context, the expression “pure component C′” means that C′ does not comprise any catalysts of component B′.

B′_{T1} is used in amounts of ≥0.6% to ≤8% by weight, preferably ≥0.8% to ≤5% by weight, particularly preferably ≥0.8% to ≤2% by weight, based on the entire first partial amount of component C′ used in step II′ (i.e. based on the total amount of C′_{T1}).

Step II′ may be carried out in such a way that the trimerization starts already during the feeding or only after the feeding of component B′. This may be thermally influenced. The first-mentioned option is preferred.

The trimerization in step II′ is carried out at reaction temperatures of preferably ≥50° C. to ≤120° C., particularly preferably ≥55° C. to ≤90° C. If HDI is used as component A′, the trimerization in step II′ is very particularly preferably carried out at ≥57° C. to ≤65° C.

In step III′, a second partial amount of catalyst component B′ (B′_{T2}) and a second partial amount of solvent component C′ (C′_{T2}) are fed to the reactor.

With regard to the way in which components B′ and C′ are fed, the statements made with respect to step II′ (paragraphs 2 to 4 under “Embodiment 2”) apply analogously here.

B′_{T2} is used in amounts of >0% to ≤0.5% by weight, preferably >0% to ≤0.1% by weight, based on the entire second partial amount of component C′ used in step III′ (i.e. based on the total amount of C′_{T2}).

The total amount of catalyst component B′ fed corresponds to the sum total of the two partial amounts from steps II′ and III′ (B′_{T1}+B′_{T2}). The total amount of catalyst component B′ fed is generally ≥0.001% to ≤2% by weight, preferably ≥0.001% to ≤1% by weight and particularly preferably ≥0.001% to ≤0.2% by weight, in each case based on the amount of isocyanate component A′ used. The total amount of solvent component C′ fed corresponds to the sum total of the two partial amounts from steps II′ and III′ (C′_{T1}+C′_{T2}). The total amount of solvent component C′ fed is preferably ≥0.3% to ≤5% by weight, particularly preferably ≥1% to ≤2% by weight, in each case based on the amount of isocyanate component A′ used.

The trimerization in step III′ is carried out at reaction temperatures of preferably ≥50° C. to ≤120° C., particularly preferably ≥55° C. to ≤90° C. If HDI is used as component A′, the trimerization in step III′ is very particularly preferably carried out at ≥57° C. to ≤65° C.

The reaction temperatures in step II′ and step III′ may be the same or different.

In step III′, the trimerization of component A′ is continued until the desired degree of trimerization is achieved.

As soon as the desired degree of trimerization is achieved in embodiment 1 or 2, the reaction may be stopped, where this may be performed chemically or thermally. Preference is given to thermal stopping. In the case of chemical stopping, the reaction solution is stopped by adding an acidic compound, an acid and/or an alkylating agent. The preferred thermal stopping is performed either at the selected reaction temperature by simple continuation of stirring after the reaction has subsided or else by increasing the temperature by up to 50° C. and continuing to stir at this temperature. Preferably, the stopping temperature is slightly increased by up to 20° C. in comparison with the reaction temperature. Particularly preferably, thermal stopping is carried out at the reaction temperature.

The processes according to the invention may be carried out as batch processes, semibatch processes or continuously, in one or more stirred tanks.

Once the trimerization reaction has ended, optionally by stopping, the reaction product present is a solution of the isocyanurate group-containing polyisocyanate P in excess monomeric di- and/or polyisocyanate of component A (reaction mixture R), or a solution of the isocyanurate group-containing polyisocyanate P′ in excess monomeric di- and/or polyisocyanate of component A′ (reaction mixture R′).

In a preferred embodiment of the invention, in a subsequent step, any monomeric di- and/or polyisocyanate of component A or A′ still present is removed from the reaction mixture R or R′ by distillation. This is preferably done by thin-film distillation under reduced pressure, for example at a pressure of below 1.0 mbar, preferably below 0.5 mbar, particularly preferably below 0.2 mbar, under very gentle conditions, for example at a temperature of 100° C. to 200° C., preferably of 120° C. to 180° C. The monomeric di- and/or polyisocyanate may be removed in a single stage, but preferably in multiple stages. For example, a falling film evaporator is used as a preliminary evaporator, in which the majority of the monomeric di- and/or polyisocyanate is removed; further starting isocyanate is removed in the downstream thin-film evaporator. In this way, high-quality isocyanurate group-containing polyisocyanates are obtained, which have a content of free monomeric di- and/or polyisocyanate of at most 0.5% by weight, preferably of at most 0.1% by weight. The distillates obtained are reused for the trimerization.

In a further embodiment, the monomeric di- and/or polyisocyanates are removed from the reaction product by extraction with suitable solvents that are inert toward isocyanate groups, for example aliphatic or cycloaliphatic hydrocarbons such as pentane, hexane, heptane, cyclopentane or cyclohexane. This process is less preferred.

The thus obtained low-monomer isocyanurate group-containing polyisocyanates are used as such or else are dissolved in suitable solvents that are inert toward NCO groups to form polyisocyanate solutions. The polyisocyanates prepared by the processes according to the invention are used in the known applications such as 2-component polyurethane coatings or in adhesive applications. As is known, the thus obtained polyisocyanates also serve as starting materials for further derivatives prepared therefrom, such as blocked polyisocyanates or hydrophilized polyisocyanates.

### Experimental Section

Unless described otherwise, reactants used were used without further purification. Hexamethylene diisocyanate and Desmodur LD were obtained from Covestro Deutschland AG. All other reactants were ordered from Sigma Aldrich (Merck AG): Triton B 40% in methanol, 2-ethylhexanol, n-butanol, 2-ethylhexane-1,3-diol, 4-heptanol and choline acetate.

The following standard methods were used:

NCO values were determined by NCO titration in accordance with M105-ISO 11909.

Viscosities were determined in accordance with M014-ISO 3219/A.3.

The free monomer content was determined by means of M106-ISO 10283.

On-line reaction monitoring was carried out using Raman spectroscopy. To this end, the RAMAN RXN2 device from Kaiser Optics was used and the associated measuring sensor was introduced into the reaction vessel and a spectrum was recorded every 2 minutes. The C═O band of the isocyanurate can be uniquely assigned to the band at wavenumber 1760 cm-1. In combination with the NCO determination by titration and with the assumption that all reacted NCO groups enter into the isocyanurate, calibration of the signals was performed.

### Apparatus Setup

The apparatus consists of a 0.5 liter jacketed glass reactor (Büchi, type 2), which can be operated up to an operating pressure of 6 bar and an operating temperature of 200° C. The reactor interior is heated via a thermostat connected to the jacket (Huber, Ministat 240 type). The temperature control in the thermostat is effected on the basis of the temperature measured in the reactor interior using a thermocouple. The most homogeneous possible mixing in the reactor is ensured using a stirrer inserted via the reactor lid. A Raman probe can be inserted into the reactor via the reactor lid and used for on-line reaction monitoring. It is also possible to take samples from the reactor using a syringe for off-line analysis.

### Batch Reaction

### Example 1: Reference Test with Conventional Metered Addition (Non-Inventive)

350 g of hexamethylene diisocyanate (HDI) was initially charged into a 0.5 l stirred reactor and heated to 70° C. in order to degas the diisocyanate at this temperature for 60 min at 20 mbar. After venting the reaction vessel and cooling the diisocyanate to 60° C., 4.6 g of a 0.5% by weight Triton B solution in 2-ethylhexanol was metered into the vessel as quickly as possible with stirring (1000 rpm). After a short waiting period, the reaction started, which led to a decrease in the free NCO value. A degree of trimerization of 10% was quickly achieved. Over the course of a further 3 hours, the NCO value continued to decrease, although at a lower decrease rate than before.

### Example 2: Use of the Described Process (Inventive)

The experiment described under Example 1 was repeated in an identical manner until a degree of trimerization of 20% was achieved. Then, 1.16 g of 2-ethylhexanol was added as one portion. After a short waiting time, the NCO value fell more sharply than in the reference experiment (Example 1). The decrease in the NCO value went beyond the extent which can be explained by the reaction of free NCO groups with the added alcohol to form urethanes and allophanates. The increased number of isocyanurate units in comparison with Example 1 was able to be confirmed using Raman spectroscopy.

Similar experiments were also carried out with n-butanol, 2-ethylhexane-1,3-diol and 4-heptanol. The combination choline acetate/2-ethylhexanol was also confirmed in the experiment.

### Example 3: The Particular Role of the Alcohol in the Comparative Experiment (Non-Inventive)

The experiment described under Example 1 was repeated in an identical manner until a degree of trimerization of 20% was achieved. Then, 5 g of Desmodur LD (Covestro sales name of 2-ethylhexyl (6-isocyanatohexyl) carbamate) was added as one portion. In comparison with Reference Example 1, the NCO value increased minimally because the added component Desmodur LD comprises free isocyanate groups. Apart from that, the course of the reaction did not change and no increased number of isocyanurate units was formed.

### BRIEF DESCRIPTION OF THE DRAWING

FIG. 1 shows a comparison of the courses of the reactions of Examples 1-3.

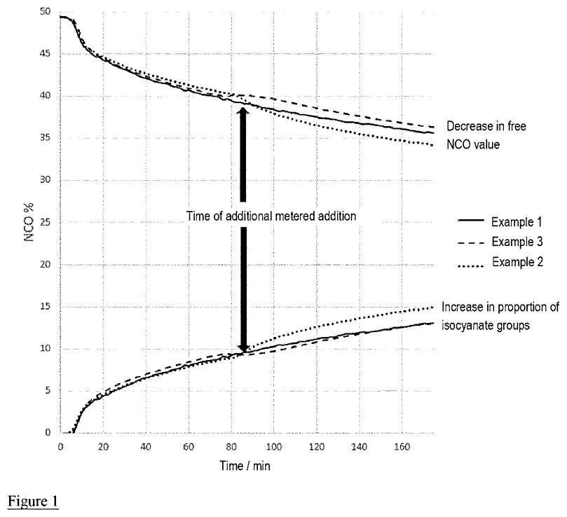

### Description of FIG. 1:

Example 1=solid line; Example 2=dotted line; Example 3=dashed line. Comparison of three trimerization reactions at 60° C. with Triton B as catalyst and 2-ethylhexanol as alcohol. The arrows indicate the time of additional metered additions for Example 2 and 3.

### DETAILED DESCRIPTION

### Semibatch Tests (Reduction of Reaction Time)

### Example 4: Semibatch Test with Conventional Metered Addition (Non-Inventive)

350 g of hexamethylene diisocyanate (HDI) was initially charged into a 0.5 l stirred reactor and heated to 70° C. in order to degas the diisocyanate at this temperature for 60 min at 1 mbar. After venting the reaction vessel and cooling the diisocyanate to 65° C., the metered addition of a 1.5% by weight Triton B solution in 2-ethylhexanol into the vessel at 0.18 ml/min was started with stirring (500 rpm). After 10 minutes, the addition of the catalyst solution was stopped. After a further 30 min, the addition of catalyst solution was resumed at 0.017 ml/min until a total of 1.85 g of catalyst solution had been metered into the vessel. The reaction was continued up to a degree of trimerization of 11.2%. The reaction was then stopped by adding DBP (dibutyl phosphate; 120% by weight in relation to the amount of Triton B used).

### Example 5: Semibatch Test Using the Described Process (Inventive)

350 g of hexamethylene diisocyanate (HDI) was initially charged into a 0.5 l stirred reactor and heated to 70° C. in order to degas the diisocyanate at this temperature for 60 min at 1 mbar. After venting the reaction vessel and cooling the diisocyanate to 65° C., the metered addition of a 1.9% by weight Triton B solution in 2-ethylhexanol into the vessel at 0.18 ml/min was started with stirring (500 rpm). After 10 minutes, the addition of the catalyst solution was stopped. After a further 30 min, at T_{g}=7.6%, the addition of pure alcohol was resumed at 0.017 ml/min until a total of 1.85 g consisting of catalyst solution and added alcohol had been metered into the vessel. The reaction was continued up to a degree of trimerization of 11.2%. The reaction was then stopped by adding DBP (dibutyl phosphate; 120% by weight in relation to the amount of Triton B used).

### Table 1: Comparison of Example 4 and 5 with Respect to Reaction Times

TABLE 1

| | 0% (start | 11.2% (target |
|---|---|---|
| | of test) | value of test) |
| Degree of | Waiting | Target value |
| trimerization | period in min | achieved after x min |
| Example 4 | 12 | 122 |
| Example 5 | 4 | 64 |

As can be seen from Table 1, in the case according to the invention the reaction starts faster and the reaction time is shortened.

The raw materials produced in Example 4 and 5 were distilled in the same way to remove the excess of free HDI monomer. As Table 2 shows, the product quality obtained is identical (as expected when using the same amounts of catalyst and alcohol).

### Table 2: Comparison of Example 4 and 5 with Respect to the Specifications of the Resins Obtained after Distillation

TABLE 2

| | Example 4 | Example 5 |
|---|---|---|
| NCO value in % | 22.8 | 22.7 |
| Viscosity/mPas @ 23° C. | 1300 | 1200 |
| Free monomer content | 0.03 | 0.04 |

1. A process for preparing isocyanurate group-containing polyisocyanates P by trimerizing
A) at least one organic di- or polyisocyanate having independently aliphatically, cycloaliphatically and/or araliphatically bonded isocyanate groups, in the presence of
B) at least one trimerization catalyst selected from the group consisting of quaternary tetraalkylammonium hydroxides, quaternary trialkylarylammonium hydroxides and hydroxyalkyl-substituted quaternary ammonium hydroxides of choline type
and
C) at least one alcohol as solvent,
comprising or consisting of the following steps
I) initially charging component A into a reactor,
II) feeding the total amount of catalyst component B, and a first partial amount of component C (C_{T1}), and trimerizing component A until a degree of trimerization T_{g} in the range of ≥0.5% to ≤25% is obtained, where
$${T}_{g} = ( {NCO}_{0} - {NCO}_{t} ) / {NCO}_{0} ,$$
where
NCO₀ corresponds to the amount of free NCO groups originally present in the initially charged component A and
NCOₜ corresponds to the amount of free NCO groups in the reaction solution at time t,
in each case determined by NCO titration in accordance with M105-ISO 11909,
III) feeding a second partial amount of component C (C_{T2}), continuing the trimerization of component A.

2. A process for preparing isocyanurate group-containing polyisocyanates P′ by trimerizing
A′) at least one organic di- or polyisocyanate having independently aliphatically, cycloaliphatically and/or araliphatically bonded isocyanate groups, in the presence of
B′) at least one trimerization catalyst selected from the group consisting of quaternary tetraalkylammonium hydroxides, quaternary trialkylarylammonium hydroxides and hydroxyalkyl-substituted quaternary ammonium hydroxides of choline type
and
C′) at least one alcohol as solvent,
comprising or consisting of the following steps
I′) initially charging component A′ into a reactor,
II′) feeding a first partial amount of catalyst component B′ (B′_{T1}), and a first partial amount of component C′ (C′_{T1}),
where catalyst component B′T is used in amounts of ≥0.6% to ≤8% by weight based on the first partial amount of component C′ (C′_{T1}) used in step II′,
and trimerizing component A′ until a degree of trimerization T_{g}′ in the range of ≥0.5% to ≤25% is obtained, where
$${T}_{g}^{′} = ( {NCO}_{0} - {NCO}_{t} ) / {NCO}_{0} ,$$
where
NCO₀ corresponds to the amount of free NCO groups originally present in the initially charged component A′ and
NCOₜ corresponds to the amount of free NCO groups in the reaction solution at time t,
in each case determined by NCO titration in accordance with M105-ISO 11909,
III′) feeding a second partial amount of catalyst component B′ (B′_{T2}), and a second partial amount of component C′ (C′_{T2}),
where catalyst component B′_{T2} is used in amounts of >0% to ≤0.5% by weight based on the second partial amount of component C′ (C′_{T2}) used in step III′,
continuing the trimerization of component A′.

3. The process as claimed in claim 1, wherein the at least one organic di- or polyisocyanate (A) is selected from the group consisting of PDI, HDI, MPDI, 1,3- and 1,4-H₆XDI, 1,3- and 1,4-XDI and NBDI.

4. The process as claimed in claim 1, wherein at least one benzyltrialkylammonium hydroxide and/or at least one hydroxyalkyl-substituted quaternary ammonium hydroxide of choline type is used as component B.

5. The process as claimed in claim 4, wherein benzyltrimethylammonium hydroxide (Triton-B) and/or 2-hydroxyethyl-trimethylammonium acetate (choline acetate) is used.

6. The process as claimed in claim 1, wherein aliphatic and/or cycloaliphatic mono- or diols are used as component C.

7. The process as claimed in claim 1, wherein catalyst component B is used in amounts of ≥0.001% to ≤2% by weight based on the amount of isocyanate component A used.

8. The process as claimed in claim 1, wherein catalyst component B is used in amounts of ≥0.3% to ≤8% by weight based on the first partial amount of component C (C_{T1}) used in step II.

9. The process as claimed in claim 1, wherein the trimerization in step II is carried out at reaction temperatures of ≥50° C. to ≤120° C. wherein the trimerization in step II′ is carried out at reaction temperatures of ≥50° C. to ≤120° C.

10. The process as claimed in claim 1, wherein the total amount of solvent component C (C_{T1}+C_{T2}) fed in step II and III is ≥0.3% to ≤5% by weight based on the amount of isocyanate component A used.

11. The process as claimed in claim 1, wherein the trimerization in step III is carried out at reaction temperatures of ≥50° C. to ≤120° C.

12. The process as claimed in claim 1, wherein the process is carried out as a batch process, semibatch process or continuously, in one or more stirred tanks.

13. The process as claimed in claim 1, wherein the trimerization of component A is chemically or thermally stopped when a desired degree of trimerization is reached.

14. The process as claimed in claim 1, wherein, in a subsequent step, any monomeric di- and/or polyisocyanate of component A still present is removed from the reaction product by distillation.

15. The process as claimed in claim 2, wherein the at least one organic di- or polyisocyanate (A′) is selected from the group consisting of PDI, HDI, MPDI, 1,3- and 1,4-H₆XDI, 1,3- and 1,4-XDI and NBDI.

16. The process as claimed in claim 2, wherein at least one benzyltrialkylammonium hydroxide and/or at least one hydroxyalkyl-substituted quaternary ammonium hydroxide of choline type is used as component B′.

17. The process as claimed in claim 2, wherein aliphatic and/or cycloaliphatic mono- or diols are used as component C′.

18. The process as claimed in claim 2, wherein catalyst component B′ is used in amounts of ≥0.001% to ≤2% by weight based on the amount of isocyanate component A′ used.

19. The process as claimed in claim 2, wherein the trimerization in step II′ is carried out at reaction temperatures of ≥50° C. to ≤120° C.

20. The process as claimed in claim 2, wherein the total amount of solvent component C′ (C′_{T1}+C′_{T2}) fed in steps II′ and III′ is ≥0.3% to ≤5% by weight based on the amount of isocyanate component A′ used.
